(19) United States
(12) Patent Application Publication
(10) Pub. No.: US 2008/0308750 A1
(43) Pub. Date: Dec. 18, 2008

(54) Method for treating a tribologically stress-resistant surface of a workpiece

(76) Inventors: Tobias Abeln, Stuttgart (DE); Oliver Kull, Ostfildern (DE)

Correspondence Address: DREISS, FUHLENDORF, STEIMLE & BECKER, POSTFACH 10 37 62, D-70032 STUTTGART, DE

(21) Appl. No.: 11/829,910

(22) Filed: Jul. 29, 2007

(30) Foreign Application Priority Data
Jul. 31, 2006 (DE) 102006036151.2.34

(51) Int. Cl. A61N 5/00

(52) U.S. Cl. 250/492.1

(57) ABSTRACT

Method for treating the surface of a workpiece with a tribologically stress-resistant surface, wherein the surface is honed in at least one step and pockets are subsequently introduced using laser structuring, wherein laser structuring is effected via ultrashort pulses having a pulse duration of less than 100 ps.

The invention concerns a method for treating the surface of a workpiece having a tribologically stress-resistant surface. A method of this type is disclosed e.g. in EP 1 275 864 B1. In order to treat the surface in such a fashion that it ensures reliable lubrication through sufficient hydrodynamic pressure even under unfavorable working conditions of parts which are movable relative to each other, a plurality of pockets are introduced into the surface, whose peripheral edges are structural edges without burs. The pockets define micropressure chambers which are suitable for receiving a lubricant.

Surfaces of this type are e.g. the cylinder bores of combustion engines. The counter elements that can move therein are the pistons. Other surfaces to be treated are e.g. bores for valves, nozzles, pumps etc., or the outer structures of end faces or outer contours.

Conventional surface treatment of this type is shown in FIG. 1. The first step consists in pre-treating, e.g. pre-honing or fine boring (FIG. 1[a]), the second step consists in a second honing process (Intermediate honing, FIG. 1[b]) which further improves the surface with regard to exactness of shape and surface quality. In a third step, the pockets 10 are introduced through so-called laser structuring, i.e. using laser radiation. Pockets of a depth of approximately 5 μm-35 μm, a length of 3 mm and a width of 20 μm-60 μm are thereby produced (FIG. 1[c]). The above-mentioned document EP 1 275 864 A1 discloses different pocket shapes. The steps which are schematically shown in FIGS. 1(a) and 1(b) can also be combined in this conventional method, thereby also obtaining the structure shown in FIG. 1(c) having burs 11, which must subsequently be removed by a deburring process (in principle also a honing process).

The production of pockets of this type using laser pulses is a highly dynamic process, wherein the material is heated, melted, evaporated and ionised within a minimum time. The pressure produced through evaporation accelerates the molten mass and forces it to the edge of the removal zone where it is partially detached, and partially solidifies as a bur. FIG. 1 shows such a bur 11.

This bur is removed in a further deburring step (FIG. 1[d]), which may also substantially be a honing step. Further material is thereby removed from the surfaces (12) that remain between the pockets 10.

FIG. 2 shows the relationships in a less schematic fashion. FIG. 2(c) after performance of laser structuring and FIG. 2(d) after final deburring/honing.

Test studies of the influence of the pulse duration on the thermal processes that take place in the workpiece have shown that a reduction in pulse duration initially reduces the thickness of the molten mass, which, however, remains nearly unchanged below 10⁻¹¹ s (10 ps) and approaches a limiting value (which depends on the fluence). An analogous saturation behavior has also been observed in connection with the evaporation time and the time until complete solidification.

This can be explained by the heating behavior on an ultrashort time scale. The laser light initially interacts with the conduction band electrons of the metal and heats them to a high temperature. A plurality of impacts is required to transfer the energy from the electrons to the atoms of the crystal lattice. The atoms are thereby heated with a considerable delay. The temperature between the electrons and the lattice atoms is balanced only after a material-specific relaxation time.

The pressure generated during evaporation by the strong volume increase accelerates the molten mass. It is thereby driven out but accumulates at the edge of the removal zone, i.e. forms a bur.

For these reasons it is still necessary to perform a deburring operation after introduction of pockets into a honed surface, i.e. post processing by a deburring operation is still required.

Consequently, post processing is always necessary. Although ns lasers (so-called short pulse lasers) are currently used to produce microstructures in the surface, it has not been possible up to now to eliminate the need for a deburring process in a subsequent step. Moreover, current laser structuring still produces deposits of molten mass in the structures, which reduce their precision.

It is therefore the underlying purpose of the invention to improve a method of the above-mentioned type.

This object is achieved in accordance with the invention with the features of claim 1.

In contrast to the described expectations, it has turned out that, with pulse durations of less than 100 ps (1 ps=1 picosecond=10⁻¹² s, 100 ps=10⁻¹⁰ s: so-called ultrashort pulse lasers) it is possible to introduce pockets through laser structuring having same dimensions as before, thereby however, preventing formation of significant bulging of molten mass. The prevention of molten mass bulging, i.e. burs 11, omits a processing step in the inventive method, i.e. the step designated in FIG. 1 with (d) (the final deburring/honing operation). Moreover, the precision of the structure is increased. This means that the pocket geometries are improved with regard to formation of edges and structure base, since there is no reduced molten bath dynamics. The laser structuring step thus represents the last processing operation. The production costs are considerably reduced, since laser structuring is produced without post processing.

FIGS. 3 and 4 explain the implementation.

FIG. 3 schematically shows the different treatment steps to obtain the desired laser structure. FIG. 4 is an enlarged and less schematic representation of the pockets 10 after treatment in accordance with FIG. 3.

The duration of ultra sort pulses is less than 100 ps (10⁻¹⁰ s). The repetition rate (frequency) must be considerably higher than in a conventional YAG laser and is in a range of a few 100 kHz. In order to obtain the same removal result as in previous laser structuring, less energy is used at the honing frequency, wherein the melt burs are minimized and can be neglected for the present accuracy and other surface requirements. The removed material is completely evaporated. The bur height is in any case smaller than the roughness of the previously finished surface 12 between the pockets 10. The process thus involves less or even no melting. The roughness of the pre-treated surface is in the range of the final quality. This is e.g. ≦3 μm RZ for cylinder tracks in combustion engines.

Lasers which generate such short pulses are currently already available (compare http://www.laserzentrum-hannover.de/kompetenzen/laserentwicklung/kurzpulslaser and https://fgsw.uni-stuttgart.de/fsb/verband/fst.html).

The beam guidance may be as described e.g. in DE 20 2004 020 494.0 or DE 20 2005 005 905.6 U.

The increased structural precision also permits smaller and flatter structures (pockets). The hydrodynamic effect and capillary effect of the pockets, which holds the lubricant, is thereby optimized. This also improves the lubrication behavior during operation and improves the reliability of lubrication, since the lubricant shows a better local retention, forms a stable lubrication film, and does not leak out of the structure.

As mentioned above, the treatment with ultrashort pulses produces no significant amount of molten mass. In consequence thereof, the graphite lamellas in the structural base are exposed during processing of lamellar grey cast iron and project past the structural base. This improves the emergency-running properties in case of inadequate lubrication during operation of surfaces that have been treated in this fashion (e.g. in engines).

Moreover, use of ultrashort pulse lasers does not or only minimally changes the structure of the treated material.

In addition to use of laser-structured piston tracks in combustion engines, this type of structuring can also advantageously be used in cases where post processing through honing of the bore is not possible, e.g. outer structuring of end faces or outer contours.

1-4. (canceled)

5. A method for treating the surface of a workpiece having a tribologically stress-resistant surface, the method comprising the steps of:
a) honing the surface to produce a surface roughness in a range of ≦3 μm RZ; and
b) laser structuring the surface following step a) to produce pockets therein, the laser structuring carried out using ultrashort pulses of less than 100 ps in duration at a frequency of 100 kHz to 10 MHz to minimize melting, wherein a height of burrs is less than a roughness of the surface produced during step a).

6. Workpiece with a surface that has been treated in accordance with the method of claim 5.
